(19) United States
(12) Patent Application Publication
(10) Pub. No.: US 2015/0094539 A1
(43) Pub. Date: Apr. 2, 2015

(54) Producing of an endoscope with an optical waveguide

(71) Applicant: Peter Eisenkolb, Tuttlingen (DE)

(72) Inventor: Peter Eisenkolb, Tuttlingen (DE)

(21) Appl. No.: 14/496,114

(22) Filed: Sep. 25, 2014

(30) Foreign Application Priority Data
Sep. 27, 2013 (DE) 102013110692.7

(51) Int. Cl. A61B 1/00; A61B 1/07

(52) U.S. Cl. CPC A61B 1/0011; A61B 1/07
USPC 600/182; 156/293

(57) ABSTRACT

In a method for producing of an endoscope, optical fibers are introduced into a lumen in the endoscope and heated, the lumen is narrowed, and the optical fibers are melted together. The narrowing of the lumen and the melting together of the optical fibers take place at least partially at the same time.

### FIELD OF THE INVENTION

The present invention relates to an endoscope with optical fibers for transmitting illumination light to the distal end of the endoscope, and to methods for producing of an endoscope.

### BACKGROUND OF THE INVENTION

In medical and technical endoscopy, illumination of the viewed object is generally necessary. To generate illumination light with a high luminous flux, use is often made of separate light sources, or of light sources integrated into the proximal end of the endoscope. The illumination light is transmitted from the proximal end to the distal end of the endoscope by means of one or more bundles of optical fibers.

There is a growing trend for the shafts of endoscopes to be made increasingly thinner. This means there is less installation space available for the optical fibers, and the outlay in terms of design and manufacture increases. Conventional designs and manufacturing methods must be called into question and modified or replaced by new ones.

### SUMMARY OF THE INVENTION

The object of the present invention is to make available an improved method for producing of an endoscope, and an improved endoscope.

This object is achieved by the subjects of the independent claims.

Developments are set forth in the dependent claims.

Embodiments of the invention are based on the concept whereby, in the producing of an endoscope, a lumen in which optical fibers are arranged is narrowed, while the optical fibers are heated to a temperature at which they are plastically deformable. By means of the lumen being narrowed, the optical fibers are melted or welded together. In the process, the optical fibers are at the same time welded to the wall or walls delimiting the lumen. The cross sections of the individual optical fibers are altered, and the spaces between the individual optical fibers are reduced. Ideally, a tight structure of optical fibers is obtained within the narrowed lumen, each with a hexagonal cross section in a honeycomb-like arrangement.

In a method for producing of an endoscope, optical fibers are introduced into a lumen in the endoscope, the optical fibers are heated, the lumen is narrowed, and the optical fibers are melted together, wherein the narrowing of the lumen and the melting together of the optical fibers take place at least partially at the same time.

The lumen has in particular a circular cross section or an annular cross section, or a cross section whose edge is composed substantially of two arcs of a circle with identical or different radii, or a cross section whose edge comprises two or more arc-shaped portions with different or identical radii, or another cross section. After the narrowing, the cross section of the lumen is in particular geometrically similar, or substantially geometrically similar, to the cross section of the lumen before the narrowing. In particular, the lumen has a straight or curved, elongate and narrow cross section, with a width varying continuously from one end to the other end, wherein it is especially the width of the cross section of the lumen that is reduced during the narrowing.

The optical fibers are in particular heated along with the wall delimiting the lumen or with the walls delimiting the lumen. The optical fibers are in particular heated by conduction of heat from a wall delimiting the lumen, wherein the wall can be heated by heat conduction or radiation or by other means. The optical fibers have in particular an inorganic non-metallic glass, another inorganic glass, another amorphous material, a polymer or another plastic. The optical fibers are in particular heated to a transformation temperature or glass transition temperature at which the material of the optical fibers has a rubber-like to viscous consistency and/or has sufficient ductility to be plastically deformed. The melting together of the optical fibers is brought about in particular by the narrowing of the lumen and the resulting mechanical pressure, although it can also begin during or after the heating and before the narrowing of the lumen, in particular on account of the wetting properties of the material of the optical fibers.

A mechanically robust structure of optical fibers in a lumen of an endoscope can be created using the method. In particular, the structure of the optical fibers has only small gaps, if indeed any. Small remaining gaps can subsequently be filled by a material that melts at lower temperatures than the material of the optical fibers. In this way, a structure can be created which is impermeable to water vapor and/or other fluids and which can in particular be suitable for the hermetically sealed closure of a volume inside the endoscope.

In a method as described here, the narrowing of the lumen and the melting together of the optical fibers take place in particular at the distal end of the endoscope.

In a method as described here, a light admission face or a light exit face on the optical fibers is formed in or on the molten area of the optical fibers.

The formation of a leaktight structure of optical fibers, either directly or, optionally, after small remaining gaps have been filled, in a method as described here, permits in particular the formation of a light exit face at the distal end of the endoscope. For this purpose, after the heating, narrowing and melting, the optical fibers are in particular cut off outside the endoscope and the cut surface is polished and optionally anti-reflection coated and/or otherwise coated. In this way, without a further transparent window component or other component, a light exit face can be created directly from the optical fibers, which face at the same time hermetically seals the endoscope and, during autoclaving, also prevents entry of moisture and/or other fluids.

Alternatively or in addition, the method can also be used at the proximal end of the endoscope, in particular for the formation of an admission face for illumination light at a coupling location where the endoscope can be coupled to a separate light source by means of a fiber-optic cable. The method can also be carried out at the proximal end of an endoscope in such a way that, without using a transparent window component or another additional component, a light admission face is created which is hermetically sealed against the entry of water vapor or other fluids.

In a method as described here, optical fibers are in particular welded to a wall delimiting the lumen.

During the narrowing of the lumen, the optical fibers are in particular welded to one or more walls delimiting the lumen. The wall or walls delimiting the lumen are in particular of a surgical stainless steel or another metallic material. The welding or cohesive joining of the optical fibers to the wall or walls delimiting the lumen can increase the mechanical robustness and can help ensure that no fluids can pass through the narrowed lumen.

In a method as described here, the optical fibers are welded in particular to an outer shaft of the endoscope.

In a method as described here, the optical fibers are welded in particular to an inner shaft of the endoscope.

The outer shaft has in particular stainless steel or another metallic material. Before the narrowing of the lumen, the outer shaft has in particular the shape of a circumferential surface or a portion of a circumferential surface of a circular cylinder. The outer shaft in particular forms a large part or by far the greatest part of the outer surface of the endoscope.

The inner shaft has in particular a stainless steel or another metallic material. The inner shaft has in particular the shape of a circumferential surface or a portion of a circumferential surface of a circular cylinder. The lumen of the inner shaft is intended in particular to receive a window component, an objective lens, a rod lens system, a camera, an ordered bundle of optical fibers and/or other means of transmitting observation light and an image or an image signal from the distal end to the proximal end of the endoscope. The inner shaft ensures in particular that the lumen lying outside the inner shaft, and provided for the optical fibers for transmission of illumination light, is screened off from the observation beam path arranged inside the inner shaft.

The inner shaft can be arranged concentrically with respect to the outer shaft, such that the lumen for the optical fibers has a circular cross section, for example. Alternatively, for example, the inner shaft can be arranged in the outer shaft such that the inner shaft touches the outer shaft at one point or in a line extending from the proximal end to the distal end.

During the narrowing of the lumen, at least either the outer shaft or the inner shaft is in particular deformed at the distal end of the endoscope. For example, the cross section of the outer shaft is reduced and/or the cross section of the inner shaft is increased.

In a method as described here, the narrowing of the lumen in particular involves two walls, which delimit the lumen, being moved relative to each other.

The lumen is delimited in particular by an outer shaft and an inner shaft, of which at least one deviates at the distal end of the endoscope from an ideally circular cylindrical shape, wherein the lumen is narrowed by moving the outer shaft relative to the inner shaft in a direction parallel or substantially parallel to an axis of symmetry of the outer shaft and/or of the inner shaft.

In a method as described here, the narrowing in particular involves a deformation of a wall that delimits the lumen.

The narrowing of the lumen in particular involves a plastic and permanent deformation of a wall that delimits the lumen.

In a method as described here, the narrowing in particular involves a crimping or curving of the wall.

The narrowing of the lumen involves in particular a crimping or a compressive plastic deformation of a distal edge area of an outer shaft or a curving or stretching plastic deformation of a distal edge area of an inner shaft.

An endoscope comprises optical fibers for transmitting illumination light to the distal end of the endoscope, wherein the optical fibers are arranged in a lumen, and wherein the optical fibers are melted together at least at one location where the lumen was narrowed during the producing of the endoscope after introducing of the optical fibers into the lumen.

The endoscope is produced in particular by means of a method as described here and has corresponding features, properties and advantages.

### BRIEF DESCRIPTION OF THE DRAWINGS

FIG. 1 shows a schematic view of an endoscope;

FIG. 2 shows a schematic cross-sectional view of the distal end of an endoscope;

FIG. 3 shows a further schematic cross-sectional view of the distal end of the endoscope from FIG. 2 during its producing;

FIG. 4 shows a schematic cross-sectional view of the distal end of a further endoscope;

FIG. 5 shows a further schematic cross-sectional view of the distal end of the endoscope from FIG. 4 during its producing;

FIG. 6 shows a further schematic cross-sectional view of the distal end of the endoscope from FIG. 4 during its producing by an alternative method;

FIG. 7 shows schematic views of a cross section of optical fibers of an endoscope;

FIG. 8 shows a schematic flow chart of a method for producing of an endoscope.

### DETAILED DESCRIPTION OF THE INVENTION

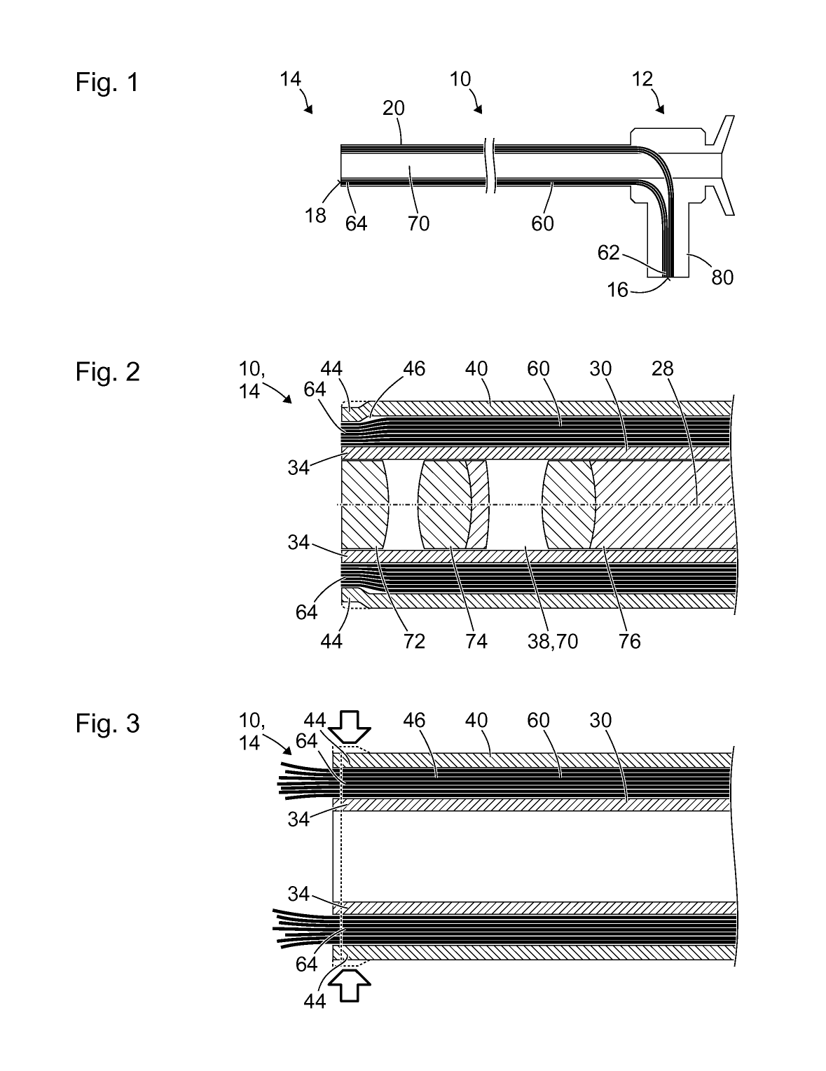

FIG. 1 shows a schematic view of an endoscope 10 with a proximal end 12 and a distal end 14, between which a long shaft 20 extends. The shaft 20 is in particular straight and rigid, although alternatively, in a departure from the view in FIG. 1, it can be at least partially curved and/or at least partially flexible. Optical fibers 60 and an observation beam path 70 are arranged in the shaft 20.

At the proximal end 12, the endoscope 10 has a coupling piece 80 for coupling the proximal end 12 of the endoscope 10 to a light source by means of a fiber-optic cable. The external light source and the fiber-optic cable are not shown in FIG. 1. A proximal end area 62 of the optical fibers 60 is arranged in the coupling piece 80 and has a light admission face 16 for illumination light transmitted from said external light source to the endoscope 10 by means of said fiber-optic cable. A distal end area 64 of the optical fibers 60 is arranged at the distal end 14 of the endoscope 10 and has there, in particular on a distal front end of the shaft 20, a light exit face 18.

The endoscope 10 is provided in particular for medical applications. The endoscope 10 should be able to be autoclaved for sterilization purposes. During the autoclaving, the endoscope 10 is exposed, for a period ranging from a few minutes to a few hours, to saturated water vapor at overpressure and at a temperature of ca. 400 Kelvin or more. Water vapor or other fluids entering the endoscope 10 may damage or destroy the endoscope 10. For this reason, the outer surface of the endoscope 10 not only has to be as smooth as possible, in order to prevent the attachment of contaminants, but also hermetically sealed, in order to prevent the introducing of water vapor and other fluids.

This also applies in particular to the light admission face 16 at the proximal end 12 and to the light exit face 18 at the distal end 14 of the endoscope 10. It will be explained below, with reference to FIGS. 2 to 8, how a smooth or substantially smooth and in particular hermetically sealed light exit face 18 can be created at the distal end 14 of the endoscope 10. A substantially smooth and in particular hermetically sealed light admission face 16 can be created in a similar way at the proximal end 12 of the endoscope 10.

FIG. 2 shows a schematic cross-sectional view of an embodiment of the distal end 14 of an endoscope 10, as described above with reference to FIG. 1. The sectional plane shown is parallel to the plane of the drawing in FIG. 1 and contains an axis of symmetry 28 of the shaft 20.

The shaft 20 comprises an inner shaft 30 and an outer shaft 40. The inner shaft 30 and the outer shaft 40 each have in particular the form of a circumferential surface or a portion of a circumferential surface of a circular cylinder and are arranged coaxially with respect to each other. The optical fibers 60 already described above with reference to FIG. 1, for transmitting illumination light from the coupling piece 80 at the proximal end 12 (cf. FIG. 1) to the distal end 14 of the endoscope 10, are arranged in the annular lumen 46 between the inner shaft 30 and the outer shaft 40.

Arranged in the lumen 38 of the inner shaft 30 is an observation beam path 70 by which light, emitted or reflected by an object to be observed, is transmitted from the distal end 14 to the proximal end 12 (cf. FIG. 1) of the endoscope 10. The observation beam path comprises in particular a light admission window 72, an objective lens 74 and a plurality of rod lenses 76, of which only one is partially indicated in FIG. 2. The light admission window 72 is joined to the distal edge area 34 of the inner shaft 30, by soldering or other means, such that the lumen 38 of the inner shaft 30 is closed in a hermetically sealed manner.

The distal edge area 44 of the outer shaft 40 is compressively deformed by crimping or other means, at least in the circumferential direction, such that the annular lumen 46 between the distal edge area 34 of the inner shaft 30 and the distal edge area 44 of the outer shaft 40 has a reduced width (measured in the radial direction) and a correspondingly reduced cross-sectional area. The optical fibers 60 in the distal end area 64 between the distal edge areas 34, 44 of the inner shaft 30 and of the outer shaft 40 are correspondingly compressed and deformed. In particular, the optical fibers 60 in the distal end area 64 are so strongly deformed, melted together or welded together, and welded to the adjoining edge areas 34, 44 of the inner shaft 30 and of the outer shaft 40, that they form a structure which has few or no pores and is therefore hermetically sealed and has a smooth surface, the latter constituting the light exit face 18 (cf. FIG. 1).

FIG. 3 shows a further schematic cross-sectional view of the distal end 14 of the endoscope 10 from FIG. 2. The sectional plane of FIG. 3 corresponds to the sectional plane of FIG. 2. In contrast to FIG. 2, FIG. 3 shows the distal end 14 of the endoscope 10 before the deformation of the distal edge area 44 of the outer shaft 40 and the resulting compression and deformation of the optical fibers 60.

In the situation shown in FIG. 3 before the compression and deformation of the optical fibers 60, the inner shaft 30 and the outer shaft 40 both have, in their distal edge areas 34, 44, the shape of a circumferential surface of a circular cylinder. After the introducing of the optical fibers 60 into the annular lumen 46 between the outer shaft 40 and the inner shaft 30, the distal edge area 44 of the outer shaft 40 is plastically and permanently deformed by a radial force acting simultaneously or in succession on the entire circumference, which force is indicated by two arrows in FIG. 3. By means of this radially acting force, the distal edge area 44 is compressed at least in the circumferential direction until it has the shape indicated in FIG. 2. In this process, the optical fibers in the area between the distal edge areas 34, 44 of the inner shaft 30 and of the outer shaft 40 are compressed and deformed.

To permit a deformation of the optical fibers 60, a melting together or welding together of the optical fibers 60, and in particular also a welding of the optical fibers 60 to the distal edge areas 34, 44 of inner shaft 30 and outer shaft 40, the optical fibers 60, and in particular also the distal edge areas 34, 44, are heated before deformation to a temperature at which the optical fibers 60 are plastically deformable. In the case of optical fibers made of glass, this temperature lies in particular within or at the transition range where the viscosity of the glass changes from brittle to liquid. In the case of optical fibers 60 made of polymers, the temperature lies in particular within the range of the glass transition temperature of the polymer or above this. In cases where the core and jacket of the individual optical fibers 60 are made of different materials, the optical fibers are heated in particular to a temperature at which the core is still substantially non-deformable but the jacket is already plastically deformable.

After the plastic deformation of the distal edge area 44 of the outer shaft 40 and of the optical fibers 60 between the distal edge areas 34, 44, the ends of the optical fibers 60 protruding beyond the distal end 14 of the endoscope 10 are cut off. Thereafter, the optical fibers 60 in the distal end area 64 and the distal end areas 34, 44 of inner shaft 30 and outer shaft 40 are ground back, in particular to a plane indicated by a broken line in FIG. 3, and are polished in order to generate a smooth light exit face 18 of high optical quality for the optical fibers 60 (cf. FIG. 1).

During the deformation of the distal edge area 44 of the outer shaft 40 and of the optical fibers 60 in their future distal end area 64 (cf. FIG. 2), and during the described grinding and polishing, the light admission window 72 and other components of the observation beam path 70 (cf. FIG. 2) are not yet inserted into the lumen 38 of the inner shaft 30. Instead, a rod of corresponding cross section can be inserted temporarily into the lumen 38 of the inner shaft 30 in order to support the distal edge area 34 of the inner shaft 30 and to prevent a plastic deformation of the inner shaft 30.

If, before the deformation, the outer shaft 40 also has the shape of a circumferential surface of a circular cylinder in its future distal edge area 44 as indicated in FIG. 3 by solid lines, it has, after the described plastic deformation, the shape indicated by solid lines in FIG. 2, with a step in its outer surface. To avoid this, the outer shaft 40 can originally be provided with an enlarged cross section, indicated by broken lines in FIG. 3, or with an increased wall thickness in its future distal edge area 44. This makes it possible, if appropriate after a final turning or grinding, to obtain a smooth and stepless outer surface of the distal edge area 44 of the outer shaft 40. The contour of such a smooth and stepless outer surface of the distal edge area is indicated in FIG. 2 by broken lines.

Alternatively or in addition to the compressive plastic deformation in at least the circumferential direction of the distal edge area 44 of the outer shaft 40 described with reference to FIGS. 2 and 3, the lumen 46 between the distal edge areas 34, 44 of inner shaft 30 and outer shaft 40 can be narrowed by a curving, or another at least circumferential plastic deformation, of the distal edge area 34 of the inner shaft 30. For this purpose, for example, before the optical elements 72, 74, 76 defining the beam path 70 are inserted into the lumen 38 of the inner shaft 30 (cf. FIG. 2), a cone with an acute aperture angle is pressed into the distal edge area 34 of the inner shaft 30 in order to widen the latter.

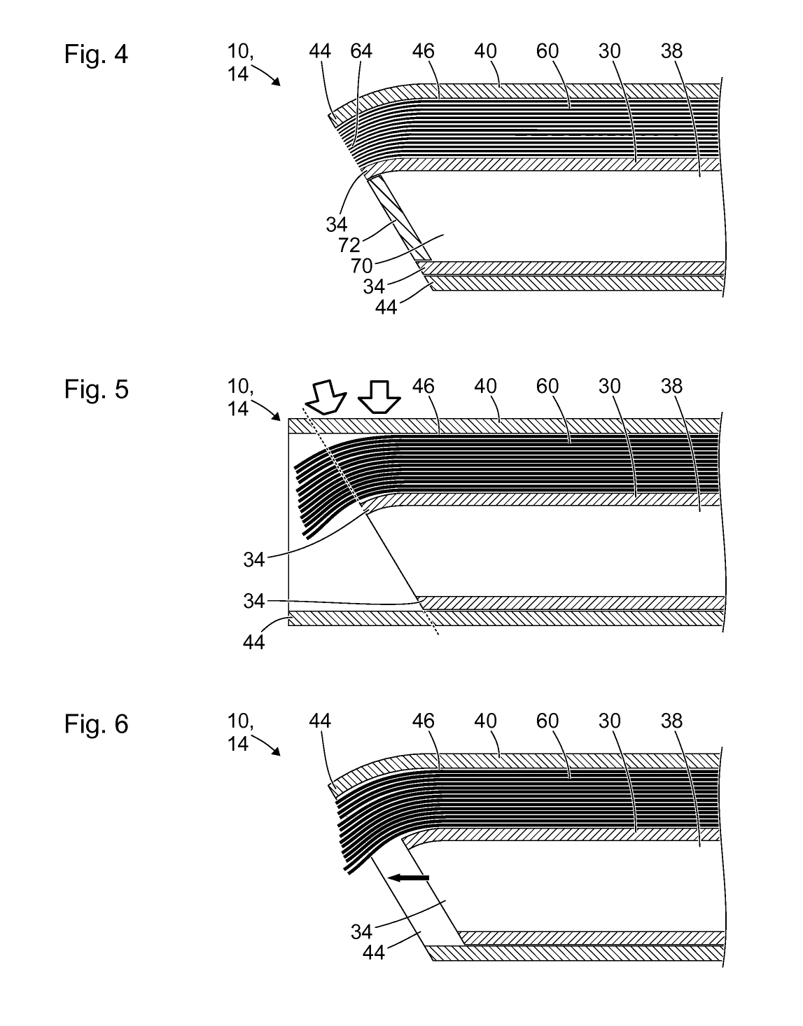

FIG. 4 shows a schematic cross-sectional view of a distal end 14 of a further endoscope 10 which, in some features and properties, is similar to the endoscopes described above with reference to FIGS. 1 to 3. The sectional plane of FIG. 4 corresponds to the sectional planes of FIGS. 2 and 3. Features and properties are described below by which the endoscope 10, of which the distal end 14 is shown in FIG. 4, differs from the endoscopes described above with reference to FIGS. 1 to 3.

The endoscope 10, of which the distal end 14 is shown in FIG. 4, differs from the endoscopes described above with reference to FIGS. 1 to 3 particularly in that it is not designed for a rectilinear viewing direction but instead for an angle of ca. 30 degrees between the viewing direction and the longitudinal axis of the shaft 20. Accordingly, the angle between the surface normal of a light admission window 72 in the observation beam path 70 and the longitudinal axis of the shaft 20 is ca. 30 degrees. Correspondingly, the light exit face 18 is also inclined, and the optical fibers 60 near the light exit face 18 are curved. In particular, the whole front end of the shaft 20 is inclined.

The endoscope 10, of which the distal end 14 is shown in FIG. 4, also differs from the endoscopes described above with reference to FIGS. 1 to 3 in that the inner shaft 30 is not arranged concentrically with respect to the outer shaft 40. Instead, the inner shaft 30 is arranged in the outer shaft 40 such that both touch each other in a straight line. In this linear area, inner shaft 30 and outer shaft 40 can be cohesively joined by soldering, welding or by other means.

In their distal end areas 34, 44, the inner shaft 30 and the outer shaft 40 are curved in such a way that not only the aforementioned curvature of the optical fibers 60 arranged in the lumen 46 between inner shaft 30 and outer shaft 40 is predefined, but also the cross-sectional area of the lumen (relative to cross-sectional areas perpendicular to the optical fibers 60) decreases in the distal direction. In this way, the optical fibers 60 are compressed in their distal end area 64.

Since the optical fibers 60 were heated, before compression, to a temperature at which they are at least partially plastically deformable and in particular can be melted together and welded to the inner shaft 30 and outer shaft 40, the optical fibers 60 are at least partially melted or welded together in their distal end area 64 and are welded to the distal edge areas 34, 44 of inner shaft 30 and outer shaft 40.

The narrowing of the lumen 46 between the distal edge areas 34, 44 of inner shaft 30 and outer shaft 40 can in particular take place in two different ways, which are set out below with reference to FIG. 5.

FIG. 5 shows a further schematic cross-sectional view of the distal end 14 of the endoscope 10 from FIG. 4. The sectional plane of FIG. 5 corresponds to the sectional plane of FIG. 4. The distal end 14 of the endoscope 10 is shown in FIG. 5 in a situation or configuration prior to the producing of the endoscope 10, and in particular prior to the narrowing of the lumen 46 between the distal edge areas 34, 44 of inner shaft 30 and outer shaft 40.

The outer shaft 40 originally has the shape of a circumferential surface of a circular cylinder indicated in FIG. 5. The inner shaft 30 is introduced into the outer shaft 40 and in particular secured there. The optical fibers 60 are introduced into the lumen 46 between outer shaft 40 and inner shaft 30. Thereafter, the outer shaft 40 is plastically deformed, as indicated by two arrows in FIG. 5, until it has the shape shown in FIG. 4. For this purpose, the outer shaft 40, in its future distal edge area 44, is compressed in particular at least in the circumferential direction. In the process, the lumen 46 between outer shaft 40 and inner shaft 30 is narrowed so far that the optical fibers 60 are compressed and, if a sufficiently high temperature has first been set, are also at least partially melted or welded together and also welded to the distal edge areas 34, 44 of inner shaft 30 and outer shaft 40. Finally, protruding areas of the outer shaft 40 and of the optical fibers 60 are cut off and ground back, to the plane indicated by a broken line in FIG. 5, and are polished in order to generate a smooth light exit face 18 of high optical quality (cf. FIG. 4).

FIG. 6 shows the distal end 14 of the endoscope 10 from FIG. 4 before manufacture by a method that represents an alternative to the method described above with reference to FIG. 5. The sectional plane of FIG. 6 corresponds to the sectional planes of FIGS. 2 to 5.

In contrast to the method described with reference to FIG. 5, it is not only the inner shaft 30 but also the outer shaft 40 that already has the intended final shape before the optical fibers 60 are introduced into the lumen 46 between outer shaft 40 and inner shaft 30. However, the inner shaft 30 is initially, and in the situation shown in FIG. 6, positioned farther proximally in relation to the outer shaft 40. It is only after complete introducing of the optical fibers 60 into the lumen 46 between outer shaft 40 and inner shaft 30 that the inner shaft 30 is moved in the distal direction relative to the outer shaft 40, as is indicated by an arrow in FIG. 6, until the intended relative position of inner shaft 30 and outer shaft 40 is reached as shown in FIG. 4. In this way, the lumen 46 between the distal edge areas 34, 44 of inner shaft 30 and outer shaft 40 is narrowed. If, during the movement of the inner shaft 30 relative to the outer shaft 40 and the narrowing of the lumen 46 between the distal edge areas 34, 44 thereof, the optical fibers have a sufficient temperature, they melt or weld at least partially together and at least partially weld to the distal edge areas 34, 44 of inner shaft 30 and outer shaft 40.

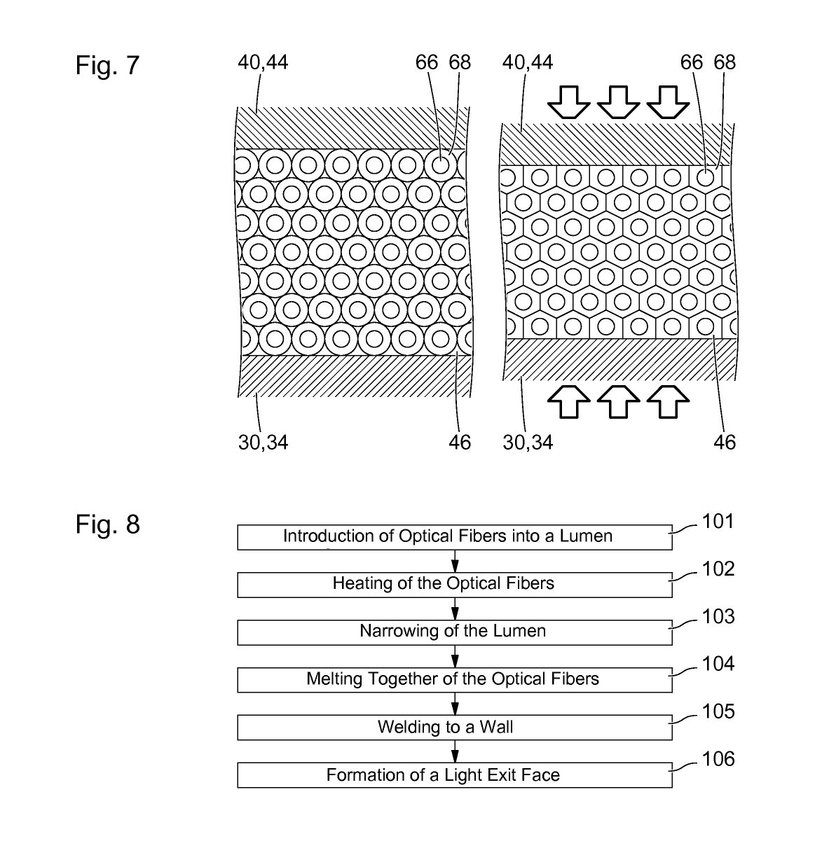

FIG. 7 shows two schematic cross-sectional views of details of the lumen 46 between the distal edge areas 34, 44 of inner shaft 30 and outer shaft 40 before (FIG. 7: left) and after (FIG. 7: right) the narrowing of the lumen 46. The sectional plane is orthogonal to the sectional planes of FIGS. 2 to 6 and orthogonal to the optical fibers 66, 68.

Each optical fiber has a core 66 and a jacket 68 made of materials with different refractive indices. Since the core 66 has a higher refractive index than the jacket 68, light is transmitted by total reflection in the core 66 of the optical fibers 60. Prior to the narrowing of the lumen 46 and the compression of the optical fibers 60 (FIG. 7: left), both the core 66 and also the jacket 68 of each optical fiber 60 have a substantially circular cylindrical shape.

After the narrowing of the lumen 46 and the deformation of the optical fibers 60 (FIG. 7: right) as a result of the forces indicated by arrows acting on the edge areas 34, 44 of inner shaft 30 and outer shaft 40, the optical fibers 60 ideally have the shape shown on the right in FIG. 7. The outer contours of the cross sections of the jackets 68 of the optical fibers 60 are hexagonal, directly adjoin one another and in particular are melted or welded together. Where the optical fibers 60 adjoin the distal edge areas 34, 44 of inner shaft 30 and outer shaft 40, they have pentagonal cross sections and are welded to the distal edge areas 34, 44 of inner shaft 30 and outer shaft 40. The optical fibers 60 form a rigid mechanical unit together with the edge areas 34, 44 of inner shaft 30 and outer shaft 40.

In the ideal case shown on the right in FIG. 7, the spaces that were present between the optical fibers 60, before the narrowing of the lumen 46 between the distal edge areas 34, 44 of inner shaft 30 and outer shaft 40 (FIG. 7: left), have completely disappeared. The structure composed of optical fibers 60 and distal edge areas 34, 44 of inner shaft 30 and outer shaft 40 is hermetically sealed.

If, in a departure from this ideal case, spaces or pores remain, for example on account of a not ideally honeycomb-shaped arrangement of the cross sections of the optical fibers 60, these gaps can later be filled with a filler material and closed. The filler material is introduced in particular in a liquid state into the gaps and hardens there, for example by polymerization. Alternatively, for example, a filler material whose melting point is lower than the melting point of the material of optical fibers 60, inner shaft 30 and outer shaft 40 is introduced in the melted state into the gaps. The filler material used can be glass solder, although it is also possible to use other suitable materials and fillers.

In the ideal case shown on the right in FIG. 7, the cores 66 of the optical fibers 60 have circular cross sections even after the compression and deformation of the jackets 68. For example, circular cross sections of the cores 66 of the optical fibers 60, even after the narrowing of the lumen 46 and the deformation of the optical fibers 60, can be obtained if, at the temperature chosen during the narrowing of the lumen 46, the material of the cores 66 has a lower plasticity than the material of the jackets 68.

FIG. 8 shows a schematic flow chart of a method for producing of an endoscope. The method is also suitable for producing of an endoscope that has features and properties deviating from those described above with reference to FIGS. 1 to 7. Notwithstanding this, reference signs from FIGS. 1 to 7 are used hereinbelow, for example, so as to make the method easier to understand.

In a first step 101, optical fibers 60 are introduced into a lumen of the future endoscope 10. The lumen is in particular a space between an outer shaft 40 and an inner shaft 30, which space can be circular or can be of any other desired shape.

In a second step 102, the optical fibers 60, along with areas 34, 44 of the future endoscope 10 adjoining them, are heated to a temperature at which the optical fibers 60 are at least partially plastically deformable, can be melted or welded together, and can be welded to the wall adjoining the lumen 46 or to the walls 34, 44 delimiting the lumen 46.

In a third step 103, the lumen 46 is narrowed. This is effected in particular by plastic deformation of at least one wall 34, 44 delimiting the lumen 46 and/or by a process in which two walls 34, 44 delimiting the lumen 46 are moved, pivoted or rotated relative to each other.

In a fourth step 104, the optical fibers 60 are at least partially melted or welded together. In a fifth step 105, the optical fibers 60 are at least partially welded to a wall 34, 44 delimiting the lumen 46. The fourth step 104 and the fifth step 105 are in particular performed at least partially during the narrowing 103 of the lumen 46 and are brought about by the narrowing 103 of the lumen 46.

In a sixth step 106, a light exit face 18 of the optical fibers 60 is generated in the area of the narrowed lumen 46.

The description with reference to FIGS. 1 to 8 has primarily explained how optical fibers 60 at the distal end 14 of an endoscope 10 are compressed by narrowing of a lumen 46 and are melted or welded together and welded to the walls 34, 44 delimiting the lumen. Correspondingly, the optical fibers 60 can be compressed at their proximal end areas 62 by narrowing of a lumen and can be melted or welded together and welded to a wall delimiting the lumen. For this purpose, the optical fibers, in their future proximal end areas 62, are in particular introduced into a metal sleeve and heated. Thereafter, the sleeve is narrowed at least locally by plastic deformation. In this way, the lumen of the sleeve in which the optical fibers 60 are arranged is narrowed and the optical fibers 60 are melted or welded together and to the sleeve. This results in a mechanically rigid structure of optical fibers and sleeve, which structure can be hermetically sealed in the ideal case described above with reference to FIG. 7, in order to protect the interior of the endoscope 10 from entry of water vapor and other fluids.

1. A method for producing of an endoscope, with the following steps:
introducing of optical fibers into a lumen in the endoscope;
heating of the optical fibers;
narrowing of the lumen;
melting together of the optical fibers,
wherein the narrowing of the lumen and the melting together of the optical fibers take place at least partially at the same time.

2. The method according to claim 1, in which the narrowing of the lumen and the melting together of the optical fibers take place at the distal end of the endoscope.

3. The method according to claim 2, also with the following step:
formation of a light admission face or of a light exit face on the optical fibers in or on the molten area of the optical fibers.

4. The method according to claim 1, in which optical fibers are welded to a wall delimiting the lumen.

5. The method according to claim 1, in which the optical fibers are welded to an outer shaft of the endoscope.

6. The method according to claim 1, in which the optical fibers are welded to an inner shaft of the endoscope (14).

7. The method according to claim 1, in which the narrowing involves two walls, which delimit the lumen, being moved relative to each other.

8. The method according to claim 1, in which the narrowing involves a deformation of a wall that delimits the lumen.

9. The method according to claim 8, in which the narrowing involves a crimping or curving of the wall.

10. An endoscope, with:
optical fibers for transmitting illumination light to the distal end of the endoscope;
wherein the optical fibers are arranged in a lumen,
wherein the optical fibers are melted together at least at a location where the lumen was narrowed during the producing of the endoscope after introducing of the optical fibers into the lumen.
